(19) United States
(12) Patent Application Publication
(10) Pub. No.: US 2011/0087310 A1
(43) Pub. Date: Apr. 14, 2011

(54) HAIR-GROWTH CARING APPARATUS

(75) Inventors: CHUNG-YANG CHEN, XINDIAN CITY (TW); CRAIG NABAT, IRVINE, CA (US)

(73) Assignee: WELLMIKE ENTERPRISE CO., LTD., TAIPEI COUNTY (TW)

(21) Appl. No.: 12/577,239

(22) Filed: Oct. 12, 2009

(51) Int. Cl. A61N 5/067

(52) U.S. Cl. 607/89

(57) ABSTRACT

A hair restoration caring device includes a treatment hood, a suspension member and a hair caring device. The treatment hood is to be disposed over a user's head. The suspension member is fastened to the treatment hood. The hair caring device is attached to the suspension member and is movable with respect to the user scalp.

### BACKGROUND OF THE INVENTION

1. Field of the Invention

The present invention relates to a hair restoration caring device, more particularly to a hair restoration caring device, in which a plurality of light emitters are applied for radiating light waves toward a user's scalp, thereby enhancing hair restoration at the balding portion of the user's scalp.

2. Description of the Prior Art

Several treatments have been proposed or developed in order to solve the baldness at human scalp due to hair loss. One method is to apply a medication lotion over the balding part of a user's scalp. The medication lotion may consist of some expander to prevent blood cell called platelets from clumping together at the terminating ends of the blood vessels, thereby enhancing blood circulation and nutrition supply to lead hair restoration at the balding part of the user's scalp. Oral suspension is recommended as another method in order to control balance of sex hormones and to prevent depauperation in cell follicles. However, there exist some side affect when oral suspension is recommended and brings unfavorable health condition to the user. Hair transplant surgery is suggested as a third method, which can restore one's initial hair style at the balding part but loss of hairs at the other part of the user's head may continue to occur. Hair grafting surgery is tremendously expensive and beyond the reach of an ordinary person.

Presently, a new hair treatment method has been developed, in which, infrared ray of 650 nm is applied to radiate at the user's scalp in order to enhance the blood circulation thereat such that blood cells are supplied with sufficient nutrition to enable hair regrowth at the balding part of the user's scalp. It is noted that the above-stated method is low in cost and does not bring side affect to the user.

Presently available infrared therapy apparatus for hair loss treatment is in a hood configuration, and generally includes a plurality of ray emitters distributed at an interior thereof. The hood must be disposed over the user's head, thereby covering the entire scalp such that the entire scalp is under the radiation of the ray emitters. The user may feel stuffy or discomfort when his entire scalp is constantly covered by the hood. In addition, the user may feel unwell if the entire scalp is put under the radiation for a long period of time.

A conventional infrared therapy apparatus is proposed, which includes a comb structure that needs to be gripped by the user's hand for disposing the ray emitters over the balding part of the user's scalp. After a period of gripping the comb structure, the user may get fatigue so that he has no desire to repeat the combing operation, thereby rendering seldom use of the conventional infrared therapy apparatus.

### SUMMARY OF THE INVENTION

Therefore, in order to solve the above-mentioned drawbacks, it is the object of the present invention is to provide a hair restoration caring device including a hair caring device movable with respect to the user's scalp so that only a specific part of the scalp will be radiated, thereby avoiding the discomfort encountered during use of the conventional infrared therapy apparatus.

The hair restoration caring device of the present invention includes a treatment hood, a suspension member and a hair caring device. The treatment hood is to be disposed over a user's head. The suspension member is fastened to the treatment hood. The hair caring device is attached to the suspension member and is movable with respect to the user scalp.

The hair restoration caring device includes an outer casing and a plurality of light emitters. The outer casing has a plurality of light passages facing toward the user's head. The light emitters are installed within the outer casing for generating and radiating light waves toward the user's scalp directly via the light passages. The hair restoration caring device is mounted slidably on the suspension member, thereby capable of radiating the light waves toward different parts of the user's head.

As explained above, the hair restoration caring device is movable with respect the user scalp and is capable of radiating only to the desired part of the scalp. There is no need for covering the entire scalp of the user's head, thereby avoiding the discomfort or stuffyness encountered during use of the conventional infrared therapy apparatus. Since only a portion of the light emitters is under operation at a time for radiating the desired part of the scalp while the remaining portion of the scalp is free from radiating, the user will not suffer from over irritation. In addition, the hair caring device is mounted by combination of the treatment hood and the suspension member so that the hair restoration caring device is not required to be held by the user's hand as in the prior art. Thus, the user of the present hair restoration caring device can do his daily or routine work without being interfered.

### BRIEF DESCRIPTION OF THE DRAWINGS

Other features and advantages of this invention will become more apparent in the following detailed description of the preferred embodiments of this invention, with reference to the accompanying drawings, in which:

FIG. 1 is a perspective view of the first embodiment of a hair restoration caring device of the present invention;

FIG. 2 shows the hair restoration caring device of the present invention disposed over a user's head;

FIG. 3 is a cross-sectional view of the hair restoration caring device of the present invention shown in FIG. 1;

FIG. 4 is a block diagram illustrating the hair restoration caring device of the present invention;

FIG. 5 shows the second embodiment of the hair restoration caring device of the present invention disposed over a user's head;

FIG. 6 is a perspective view of the second embodiment of the hair restoration caring device of the present invention;

FIG. 7 is a cross-sectional view of the second embodiment of the hair restoration caring device of the present invention shown in FIG. 6; and

FIGS. 8 and 9 respectively show the second embodiment of the hair restoration caring device of the present invention disposed over the user's head in two conditions.

### DETAILED DESCRIPTION OF THE PREFERRED EMBODIMENTS

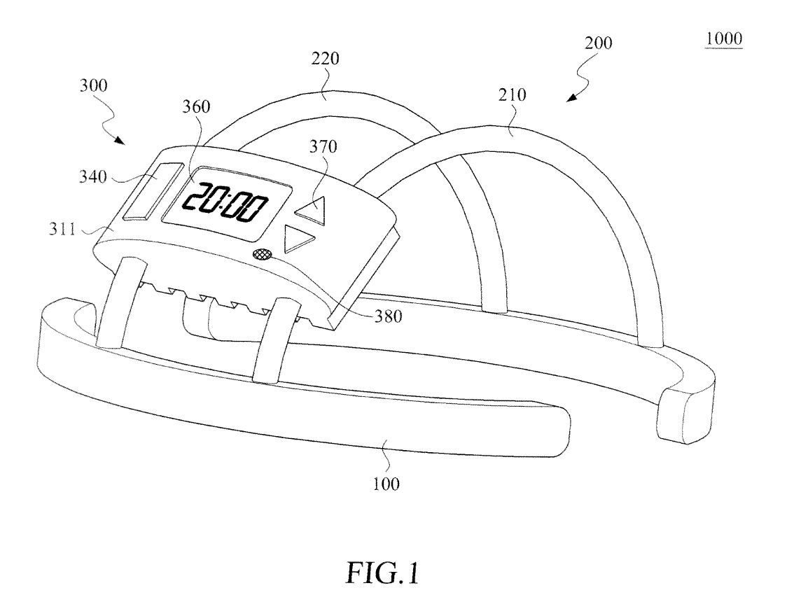

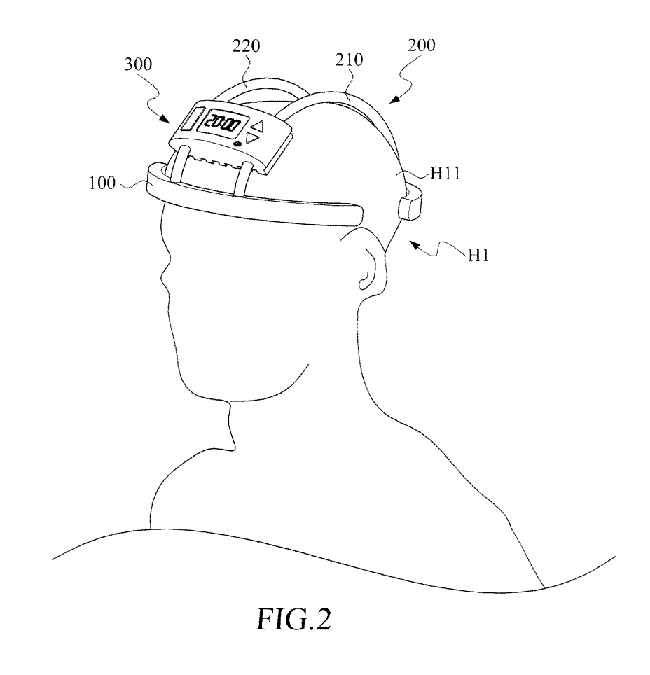

FIG. 1 is a perspective view of the first embodiment of a hair restoration caring device 1000 of the present invention while FIG. 2 shows the hair restoration caring device 1000 of the present invention disposed over a user's head. As illustrated, the hair restoration caring device 1000 accordingly includes a treatment hood 100, a suspension member 200 and a hair caring device 300.

The treatment hood 100 is an adjustable curved structure and is adapted to be disposed over the user's head H1, as best shown in FIG. 2.

The suspension member 200 is fastened to the treatment hood 100 while the hair caring device 300 is attached to the suspension member 200. In this embodiment, the suspension member 200 is fastened securely to the treatment hood 100 while the hair caring device 300 is attached slidably on the suspension member 200.

The suspension member 200 includes first and second suspending rails 210, 220, in the form of rod structure. The first and second suspending rails 210, 220 are curved ergonomically so as to span over the top part H11 of the user's head H1. The first and second suspending rails 210, 220 have first and second ends fastened securely to the treatment hood 100 in such a manner that the first and second suspending rails 210, 220 extend through the hair caring device 300. Under this condition, the hair caring device 300 is slidable on the first and second suspending rails 210, 220, and is thus movable from a front portion of the top part H11 toward a rear portion of the top part H11 of the user's head H1.

In another embodiment, the suspension member 200 includes only the first suspending rail 210 and excluding the second suspending rail 220.

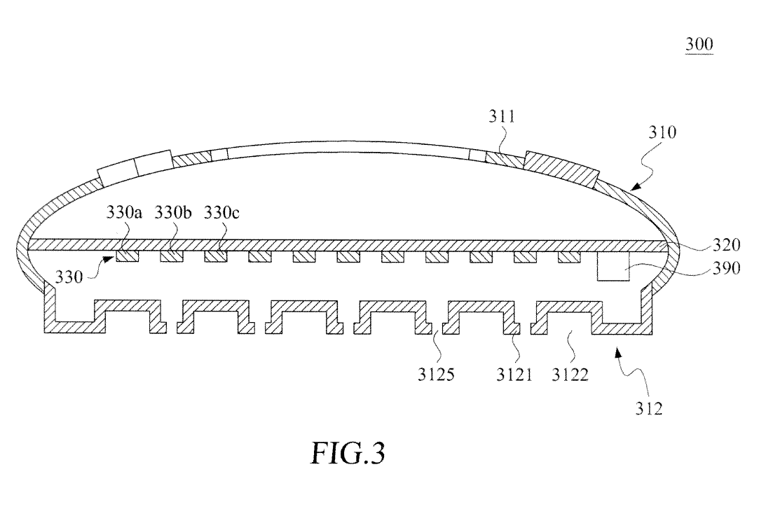

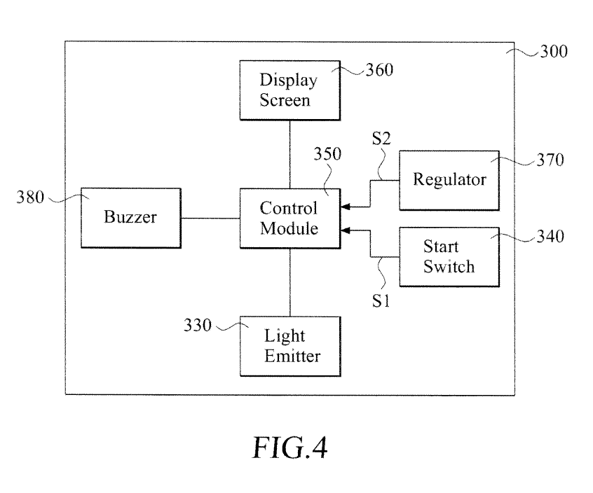

Referring again to FIGS. 1 to 4, wherein FIG. 3 is a cross-sectional view of the hair restoration caring device of the present invention shown in FIG. 1 while FIG. 4 is a block diagram illustrating the hair restoration caring device of the present invention. The hair caring device 300 further includes an outer casing 310, a plurality of light emitter 330, a start switch 340, a display screen 360, a regulator 370 and a buzzer 380.

As illustrated in FIG. 3, the outer casing 310 is a hollow structure and has an upper portion 311 and a lower transparent portion 312 facing toward the user's head H1. The transparent portion 312 of the outer casing 310 is formed with a plurality of downward projections 3121 and a plurality of upward recessions 3122 spaced apart and alternately disposed from one another. Each of the downward projections 3121 is formed with a light passage 3125 to permit passage of light therethrough. A printed circuit board 320 is mounted within the outer casing 310.

Each of the light emitters 330 can be an LED (Light Emitting Diode) and is capable of emitting light wave for radiating toward the user's head H1 via the transparent portion 312 of the outer casing 310. The light emitters 330 are mounted on the printed circuit board 320 and are directed toward the transparent portion 312 of the outer casing 310. In this embodiment, each of the downward projections 3121 is aligned with a respective one of the light emitters 330 so that the light wave can be radiated toward the user's scalp H1 through the light passage 3125 in the respective downward projection 3121. The downward projections 3121 and upward recessions 3122 cooperatively serve the purpose of hair combing operation.

In the present embodiment, each light wave ranges from 600 nm to 1000 nm, which irritates cell follicles at the balding part of user's scalp so that hair regrowth at the balding part is possible. Thus, the hair restoration caring device of the present invention is capable of performing hair restoration at the balding part of the user's scalp. The light emitters 330 consist of a plurality of first light emitters 330a, a plurality of second light emitters 330b and a plurality of third light emitters 330c. Each of the first and second light emitters 330a, 330b is an LED (Light Emitting Diode) while each of the third light emitters 330c is a laser module. Note that each of the first, second and third light emitters 330a, 330b, 330c is arranged stagger to one another and generates light wave different from one another. For instant, the light wave generated by the first light emitter 330a ranges from 700 nm to 1000 nm and in this embodiment is 880 nm. The light wave generated by the second light emitter 330b ranges from 600 nm to 7000 nm, and in this embodiment is 660 nm. The light wave generated by the third light emitter 330c ranges from 600 nm to 7000 nm, and in this embodiment is 660 nm. When the balding part of the user's scalp is irritated by different light waves, hair regrowth at the balding part is further possible.

Referring again to FIG. 1, the start switch 340, the display screen 360, the regulator 370 and the buzzer 380 are mounted on the upper portion 311 of the outer casing 310. The start switch 340 is provided for activating the plurality of light emitters 330. The display screen 360 is intended for displaying a time set for operation of the plurality of light emitters 330 while the regulator 370 is used for regulating an amount of the time set for operation of the plurality of light emitters 330. The buzzer 380 is capable of generating an alarm at a predetermined time interval to remind the user.

Referring again to FIG. 4, the hair restoration caring device further includes a control module 350, preferably a microprocessor, coupled electrically to the start switch 340, the display screen 360, the regulator 370 and the buzzer 380 in such a manner that activation of the start switch 340 generates an ON signal S1 transmitted to the control module 350. Upon receipt of the ON signal S1, the control module 350 will activate the light emitters 330 to emit light waves and the display screen 360 displays the set time for operation of the light emitters 330 (or radiating time), thereby counting the set time to the last minute. In FIG. 1, the operation time of the light emitters 330 is [20:00], i.e., 20 minutes. Thus, when 1 minute passed by, the control module 350 adjusts the set time as [19:59], and upon reaching the set time [00:00], the control module 350 will de-activate the light emitters 330 and electrical supply to the display screen 360 is simultaneously disconnected. At this time, the buzzer 380, being connected to the control module 350, gives out an alarm at a predetermined time interval, reminding the user of the hair restoration caring device of the present invention that radiation of the light emitters 330 is approaching the set time. For instant, if the time interval is set at 5 minutes, that is [15:00], [10:00], [5:00] and [0:00], the control module 350 will activate the buzzer 380 to give out alarm at 5 minute interval and the ending alarm at [0:00] is significantly different from the other, informing the user completion of application of the hair restoration caring device of the present invention.

The user can adjust the set time with assistance of the regulator 370. When the regulator 370 is rotated to set the desired time, an adjust signal S2 is transmitted to the control module 350. Upon receipt of the adjust signal S2, the control module 350 will display the radiation time over the display screen 360. For instant, the radiation time is adjusted from [20:00] to [15:00], i.e., the radiation time is set for 15 minutes.

The present hair restoration caring device further includes a sensor unit 390 connected electrically to the control module 350 for detecting presence of the treatment hood 100 over the user's head H1. The sensor unit 390 is an infrared sensor device coupled electrically to the hair caring device 300 in such a manner that the sensor unit 390 may generate infrared ray for detecting light waves emitted by the light emitters 330, thereby ensuring presence of the hair restoration caring device over the user's head H1. In case, the sensor unit 390 is unable to detect the light waves emitted by the light emitters 330, a stop signal is generated and transmitted to the control module 350, which, in turn, de-activate the light emitters 330. In the event, the sensor unit 390 detects the light waves of the light emitters 330; the control module 350 will activate the light emitters 330 to precede the operation thereof until to the set time.

The sensor unit 390 is installed on the treatment hood 100, the suspension member 200 or within the hair caring device 300, the installation position thereof is not restricted in the present invention.

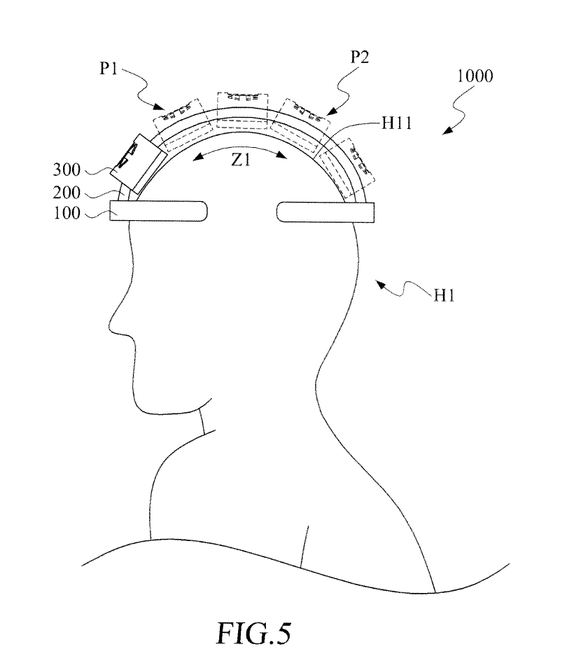

Referring to FIG. 5, the hair caring device 300 is arranged to shift from one spot to the other on the user's head H1 upon generation of each alarm so as to radiate the different parts of the balding scalp H11. Generally speaking, balding usually occurs at two sides of the forehead near the ears, beginning from the front part to the rear part so that balding due to hair loss at the scalp is easily visible.

When the hair restoration caring device 1000 is applied, those part near the ears is not required for radiation so that the overall weight thereof can be reduced. Since balding in one person differs from the other, radiation should be concentrated to the balding part of the user scalp. For instant, the hair caring device 300 is shifted between the positions P1 and P2 in order to concentrate the radiation onto the part Z1 as shown in FIG. 5. Since the hair caring device 300 of the present invention can be arranged to concentrate only on the desired balding part of the user scalp, the problem of covering the entire head and causing discomfort as encountered in the prior art can be avoided. In other words, the non-radiated part or the non-required part of the balding part of the user scalp get full rest during radiation to the desired balding part of the user scalp, irritation or over irritation at the user scalp can be avoided.

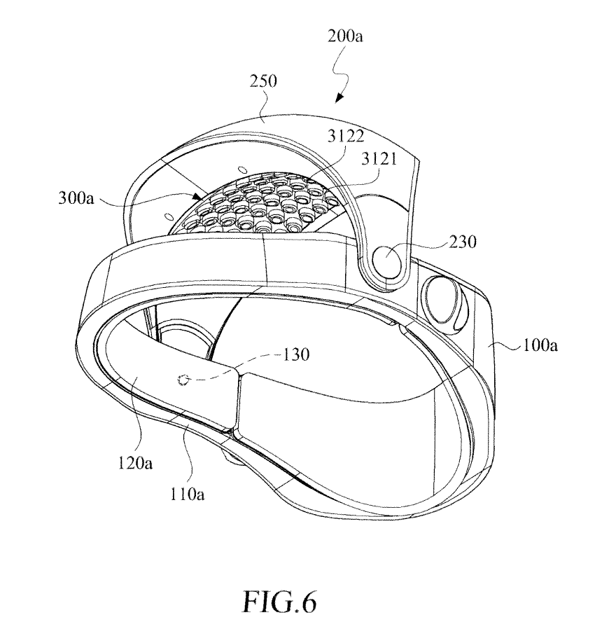

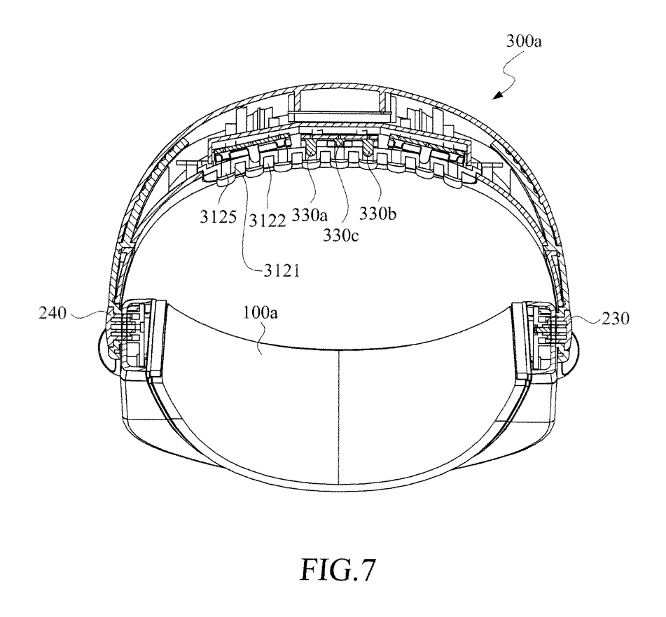

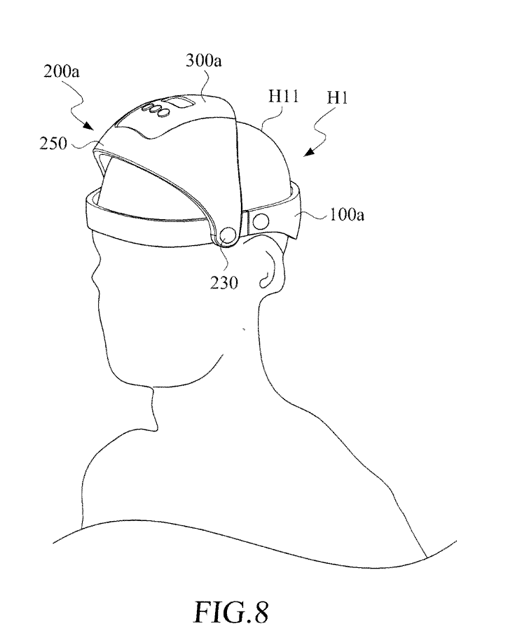

Referring to FIGS. 6 to 8, wherein FIG. 6 is a perspective view of the second embodiment of the hair restoration caring device of the present invention, FIG. 7 is a cross-sectional view of the second embodiment of the hair restoration caring device of the present invention shown in FIG. 6 while FIG. 8 shows the second embodiment of the hair restoration caring device of the present invention disposed over the user's head. The only difference with respect to the previous embodiment resides in that the suspension member 200a of the second embodiment includes a safety portion 250 (see FIG. 7) having first and second ends 230, 240 pivotally connected to two symmetric sides of the safety portion 250 (left and right sides of the user's head H1) such that the latter span over the top part H11 of the user's head H1. Note that the hair caring device 300a is installed interior to the safety portion 250 (see FIG. 6) in such a manner to face one lateral side of the user's head H1 with the downward projections 3121 directing toward the lateral side in an array manner.

The second embodiment further includes a forehead protection member 120a generally made from elastomeric material and fixed within the outer casing 110a to provide and cushion the user's forehead when impacted and a resilient switch button 130 installed within the outer casing 110a.

The resilient switch button 130 is electrically connected to the hair caring device 300 in such a manner that the control module 350 de-activates the light emitters 330 when no external force is applied on the resilient switch button 130. In other words, when an external force is applied on the switch button 130 due to wearing of the apparatus over the user's head H1, the control module 350 activate the light emitters 330 to emit light waves therefrom for radiating onto the balding part of the user scalp. The resilient switch button 130 can be substituted by the sensor unit 390 of the previous embodiment. In the same manner, the sensor unit 390 of the previous embodiment can be replaced by the resilient switch button 130 of the second embodiment.

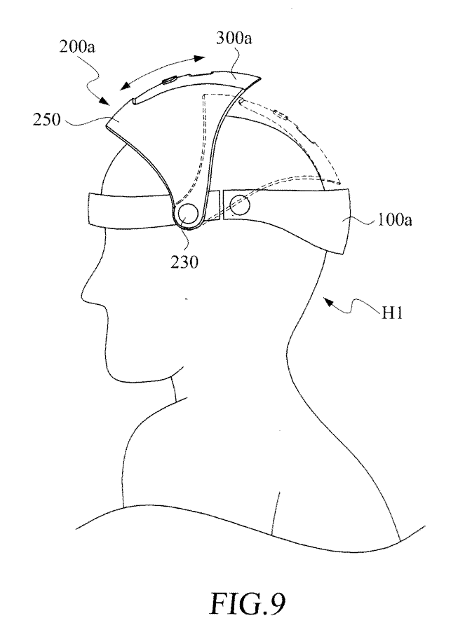

FIG. 9 shows the second embodiment of the hair restoration caring device of the present invention disposed over the user's head in another condition, wherein the user can rotate the suspension member 200a with respect to the treatment hood 100 so as to shift the hair caring device 300a from one place to the other for radiating the light waves to different parts of the user balding scalp.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A hair restoration caring device comprising:
a treatment hood for disposing over a user's head;
a suspension member fastened to said treatment hood; and
a hair caring device attached to said suspension member and including
an outer casing having a plurality of light emitter installed within said outer casing for generating and radiating light waves toward the user's scalp.

2. The hair restoration caring device according to claim 1, wherein said treatment hood is an adjustable curved structure adapted to be disposed over the user's head.

3. The hair restoration caring device according to claim 1, wherein said suspension member is fastened securely on said treatment hood while said hair caring device is mounted slidably on said suspension member.

4. The hair restoration caring device according to claim 1, wherein said hair caring device is mounted slidably on said suspension member, thereby radiating said light waves toward different parts of the user's head.

5. The hair restoration caring device according to claim 4, wherein said suspension member includes a first suspending rail extending through said hair caring device with said hair caring device mounted slidably on said first suspending rail.

6. The hair restoration caring device according to claim 5, wherein said suspension member further includes a second suspending rail extending through said hair caring device with said hair caring device mounted slidably on said second suspending rail.

7. The hair restoration caring device according to claim 6, wherein said first and second suspending rails are curved so as to span over the user's head.

8. The hair restoration caring device according to claim 1, wherein said suspension member has first and second ends connected pivotally to said treatment hood.

9. The hair restoration caring device according to claim 8, wherein said first and second ends of said suspension member are connected pivotally to two symmetric sides of said treatment hood.

10. The hair restoration caring device according to claim 1, wherein said hair caring device is slidable on said suspension member and is movable from a front portion toward a rear portion of the user's head.

11. The hair restoration caring device according to claim 1, wherein said hair caring device includes:
a display screen installed on said outer casing for displaying a time set for operation of said plurality of light emitters; and
a regulator for regulating an amount of said time set for operation of said plurality of light emitters.

12. The hair restoration caring device according to claim 11, wherein said hair caring device further includes a start switch mounted on said outer casing for activating said plurality of light emitters.

13. The hair restoration caring device according to claim 11, wherein said hair caring device further includes a buzzer for generating an alarm at a predetermined time interval.

14. The hair restoration caring device according to claim 11, wherein said transparent portion of said outer casing is located at a lower part thereof and is formed with a plurality of downward projections spaced apart from one another.

15. The hair restoration caring device according to claim 14, wherein each of said downward projections is aligned with a respective one of said light emitters in said outer casing.

16. The hair restoration caring device according to claim 1, wherein each of said light waves ranges from 600 nm to 1000 nm.

17. The hair restoration caring device according to claim 1, wherein said light emitters includes a plurality of first light emitters, a plurality of second light emitters and a plurality of third light emitters of said first, second and third light emitters generating light wave different from one another.

18. The hair restoration caring device according to claim 17, wherein said light wave generated by said first light emitter is 880 nm, said light wave generated by said second light emitter being 660 nm and said light wave generated by said third light emitter being 650 nm.

19. The hair restoration caring device according to claim 17, wherein each of said first and second light emitters is an LED (Light Emitting Diode) while each of said third light emitters is a laser module.

20. The hair restoration caring device according to claim 1, further comprising a resilient switch button connected electrically to said hair caring device in such a manner that said plurality of light emitters are de-activated when no external force is applied onto said resilient switch button.

21. The hair restoration caring device according to claim 20, wherein said resilient switch button is installed within said treatment hood.

22. The hair restoration caring device according to claim 1, further comprising a sensor unit for detecting presence of said treatment hood over the user's head.

23. The hair restoration caring device according to claim 22, wherein said sensor unit is coupled d electrically to said hair caring device in such a manner that said sensor unit is capable of de-activating said light emitters upon detecting absence of said hair caring device.
